(19) United States
(12) Patent Application Publication
(10) Pub. No.: US 2021/0227770 A1
(43) Pub. Date: Jul. 29, 2021

(54) CANNABIS PLANT NAMED 'FRB005'

(71) Applicant: FRONT RANGE BIOSCIENCES, INC., Lafayette, CO (US)

(72) Inventors: Jeff Yellin, Westminster, CO (US); Jonathan David Vaught, Longmont, CO (US); Christopher Zalewski, Seaside, CA (US); Ray French, Fairhope, AL (US)

(21) Appl. No.: 17/103,548

(22) Filed: Nov. 24, 2020

Related U.S. Application Data
(63) Continuation of application No. 16/873,064, filed on Jan. 23, 2020, now abandoned.

(51) Int. Cl. A01H 6/28; A01H 5/12

(52) U.S. Cl. CPC A01H 6/28; A01H 5/12

(57) ABSTRACT

The unique annual herbaceous Cannabis plant variety C. sativa ‘FRB005’ is provided.

### CROSS REFERENCE TO RELATED APPLICATION

This application is a continuation application of U.S. application Ser. No. 16/873,064, filed on Jan. 23, 2020, which is fully incorporated by reference herein.

### LATIN NAME OF THE GENUS AND SPECIES

Genus—Cannabis.

Species—sativa.

### VARIETY DENOMINATION

The new Cannabis plant claimed is of the variety denominated ‘FRB005’.

### BACKGROUND OF THE INVENTION

The present invention relates to a new and distinct hybrid hemp plant, which has been given the variety denomination of ‘FRB005’. ‘FRB005’ is derived from selfed seed of a variety comprised of the two subspecies of Cannabis sativa. The two primary Cannabis subtypes, hemp and marijuana are primarily distinguished by the amount of phytochemicals they contain, in particular delta-9-tetrahydrocannabinol (i.e., THC) and cannabidiol (i.e., CBD). Hemp contains a very low concentration of THC, while marijuana often has concentration of THC in excess of about 10%. The cutoff between hemp and marijuana, in terms of THC levels, as defined by Congress in 2018, is 0.3% THC: cultivars that yield a lower amount of THC can be classified as “Industrial Hemp” while others with higher amounts of THC are classified as marijuana. These measurements are based upon the weight percentage of the cannabinoid in question as a function of the total dry weight of flower.

The plant disclosed herein was discovered when the inventors were intentionally self-pollinating and cultivating plants in Boulder, Colo. to derive improved varieties of hemp. The self-pollination was done using the female variety both as a female, and reversed as a male for pollen production. Initial evaluation of seedlings from selfing was done at the indoor growing facility in Boulder, Colo., followed by 2020 variety trial and production evaluation at multiple field locations around the USA (VT, PA, VA, NC, AL, OH, WI, CO, and CA).

The clonal plants produced large numbers of 51 seeds which were germinated in a greenhouse and tested under typical hemp growing conditions indoors in 2019 and in the field in 2020.

### SUMMARY OF THE INVENTION

The novel seed variety designated ‘FRB005’, is self-seed of the clonal variety ‘Indoor Angie’ (not patented), an internal variety. ‘Indoor Angie’ originated from a seed of the female parent ‘River Rock’ (not patented), and the male source of pollen is unknown. ‘Indoor Angie’ was the 11^{th} seedling selected for evaluation, and was easily propagated and maintained plant traits consistently through multiple generations of clonal propagation.

‘FRB005’ exhibits the following characteristics: (a) pest and disease tolerance; (b) a strong smoky odor characteristic of certain terpenes and terpene derivatives such as thiols, and well-liked by growers for potential smokable flower; (c) a high percentage of CBD; (d) primarily early maturity flowering, with 25% super early flowering plants and 75% early flowering plants and (e) primary flowers that are at least 15.3-30.5 cm in length for the majority of the seedlings (Table 1).
High CBD level of at least 10%, e.g., in the range of 12-20% (in dry flower);
High CBD/THC ratio of at least 24:1, e.g., in the range 20:1 to 40:1 (in dry flower);
High terpene level, e.g., one or more of Limonene, Valencene, Linalool, Bisabolol, Geraniol and Terpineol; and
Greater than 99% females.

### BRIEF DESCRIPTION OF PICTURES

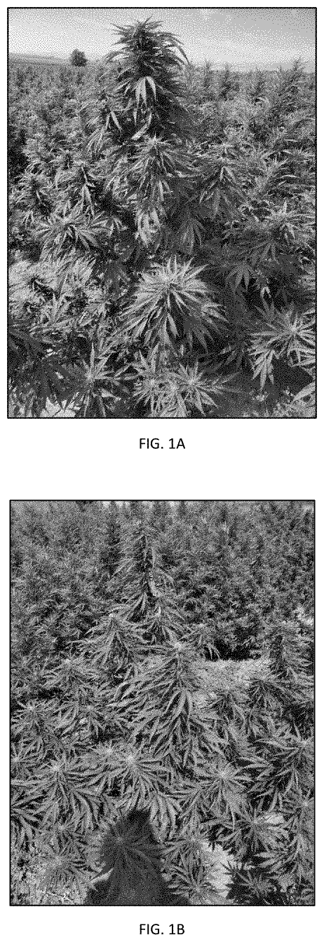

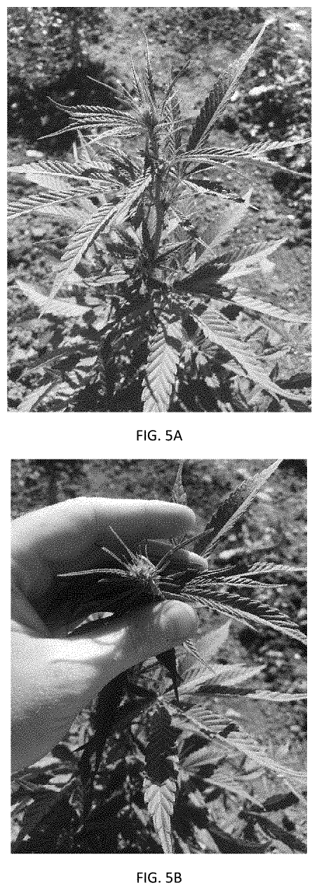

The accompanying photographs illustrate the overall appearance of the new variety showing the colors as true as it is reasonably possible to obtain in colored reproductions of this type. Colors in the photographs may differ slightly from the color values cited in the detailed botanical description which accurately describe the colors of the new hemp variety. These photographs show the colors as true as it is reasonably possible to obtain in reproductions of this type. Colors in the photographs may differ slightly from the color values cited in the detailed botanical description which accurately describe the colors of the new plant. The accompanying indoor photographs illustrate the overall appearance of the clonal parent variety (FIGS. 1-4) vs. the new plant inbred seed variety claimed (FIGS. 5-8).

FIG. 1A and FIG. 1B each depicts a single plants of C. sativa clonal ‘Indoor Angie’, parent of inbred seed variety ‘FRB005’, just south of Longmont, Colo. (1505 meters elevation, 40.145285 latitude, −105.075615 longitude) on Sep. 3, 2019.

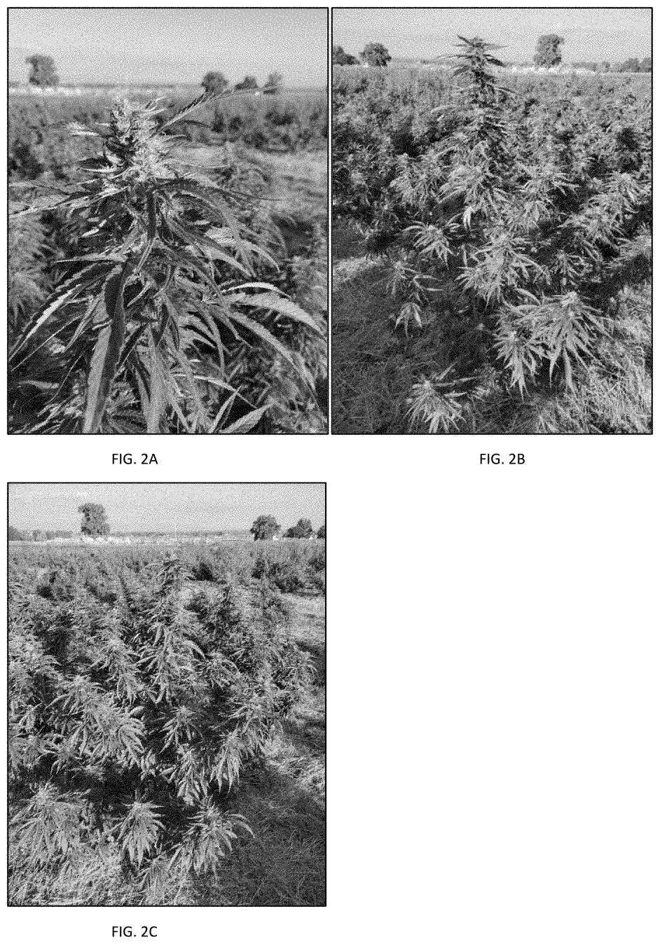

FIG. 2A depicts a close-up view of a typical inflorescence and FIG. 2B and FIG. 2C each depicts a single plant of the C. sativa clonal ‘Indoor Angie’, parent of inbred seed variety ‘FRB005’ at Frederick, Colo. (1495 meters elevation, 40.132407° N, −104.982562° W) on Sep. 11, 2019.

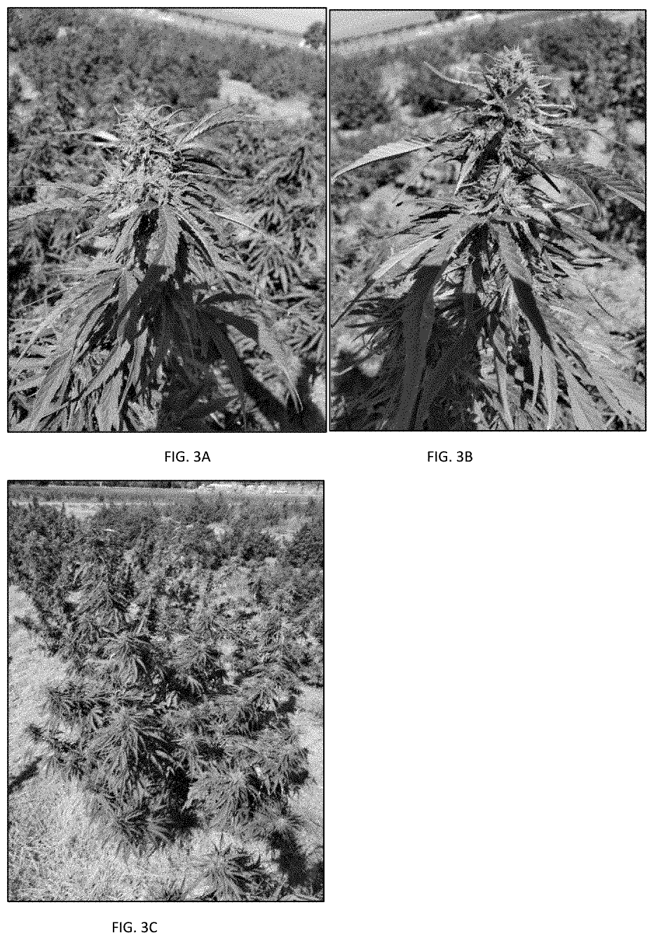

FIG. 3A and FIG. 3B each depicts a close-up view of a typical inflorescence and FIG. 3C depicts a single plant of C. sativa clonal ‘Indoor Angie’, parent of inbred seed variety ‘FRB005’ at Frederick, Colo. on Sep. 25, 2019.

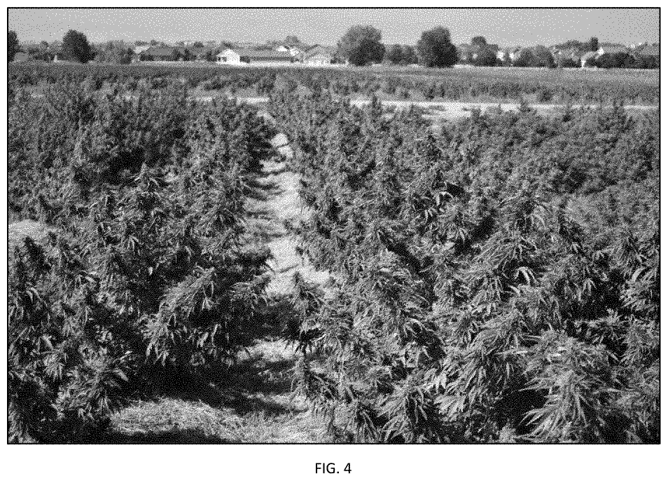

FIG. 4 depicts a small production area of C. sativa clonal ‘Indoor Angie’, parent of inbred seed variety ‘FRB005’ at Frederick, Colo. on Sep. 25, 2019.

FIG. 5A and FIG. 5B each depicts young buds of inbred seed variety ‘FRB005’, part of the 20-25% super early flowering/maturing seedlings, on Jul. 23, 2020 west of Longmont, Colo. in the foothills of the Rocky Mountains (1641 meters elevation, 40.123197° N, −105.231653° W). A similar 25% super early plants of ‘FRB005’ was seen at Frederick, Colo.

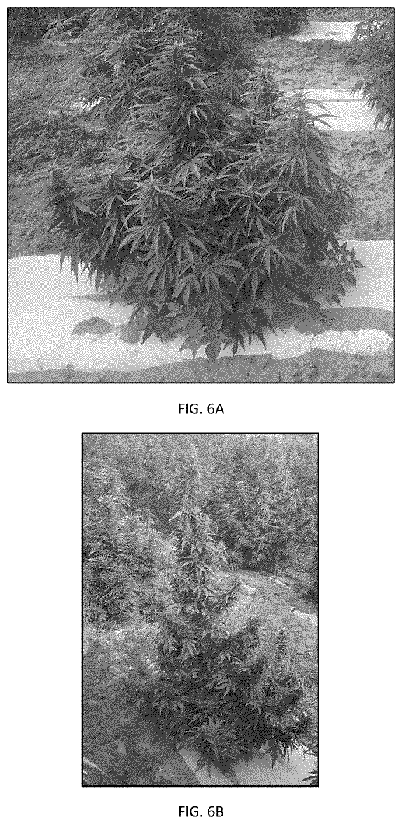

FIG. 6A depicts a short ‘FRB005’ phenotype (2′ tall) still resembling the clonal parent ‘Indoor Angie’. FIG. 6B depicts shorter branches than the typical clonal parent ‘Indoor Angie’, in a ‘FRB005’ phenotype that is 90 cm tall. FIG. 6C depicts a single ‘FRB005’ plant with a greater number of branches and a branch height that is more even with the mainstem than typically found in the clonal parent ‘Indoor Angie’. FIG. 6D depicts ‘FRB005’ flowering.

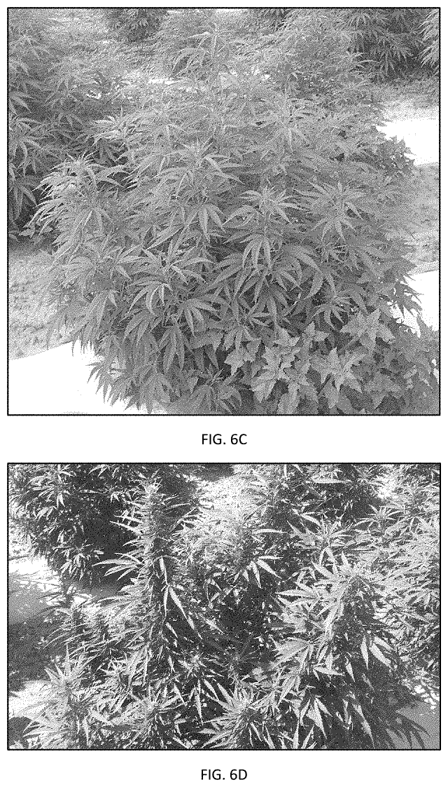

FIG. 7A depicts upper petiole color rating (RHS Colour Chart 77A), and FIG. 7B depicts 3 types of variance for purple, upper petiole color from 3 phenotypes of ‘FRB005’.

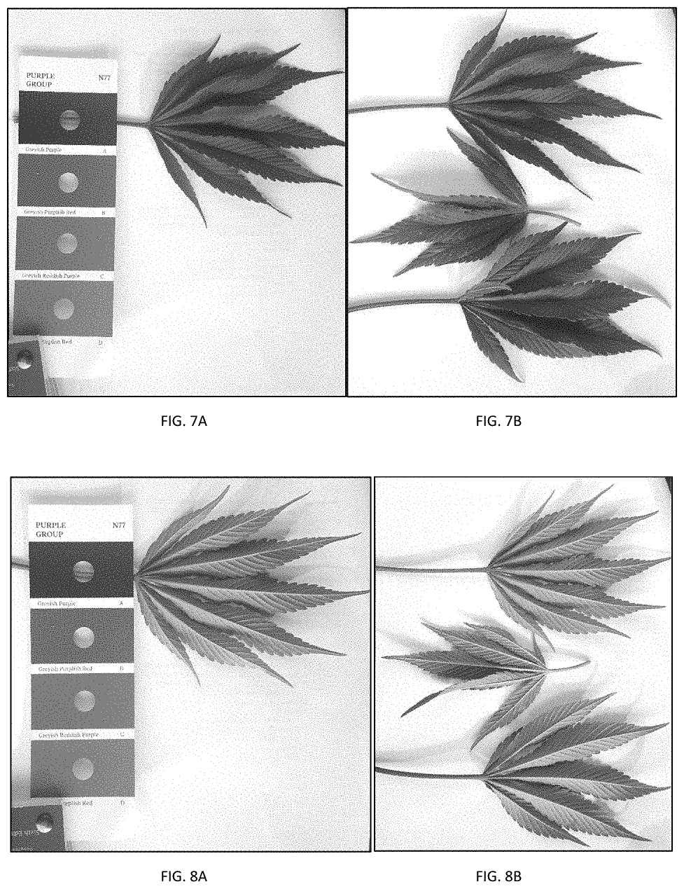

FIG. 8A depicts underside petiole color rating (RHS Colour Chart 77A), and FIG. 8B depicts 3 types of variance for purple, underside petiole color from 3 phenotypes of ‘FRB005’.

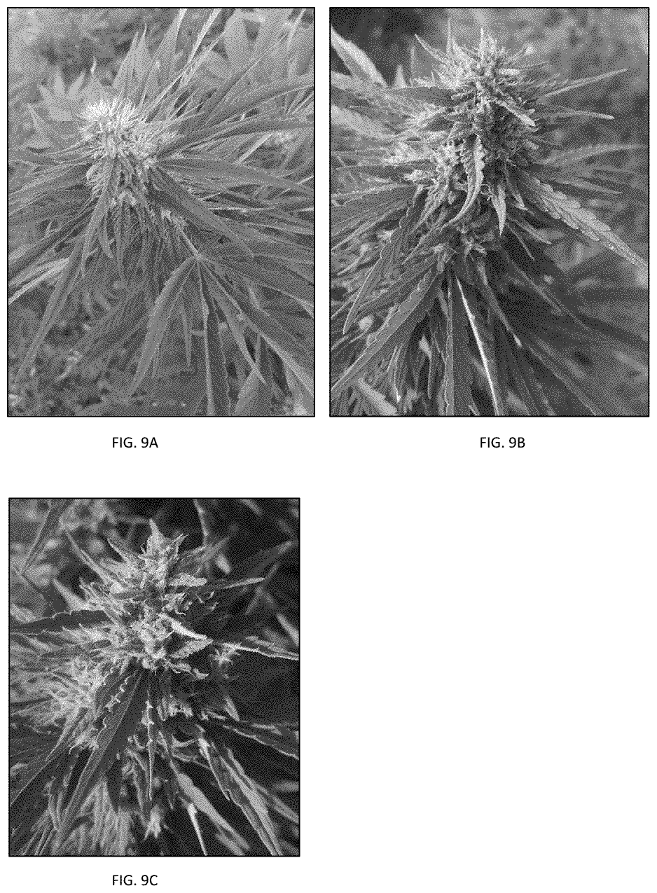

FIG. 9A depicts typical white flower of ‘FRB005’ and FIG. 9B and FIG. 9C each depicts rare purple flower ‘FRB005’.

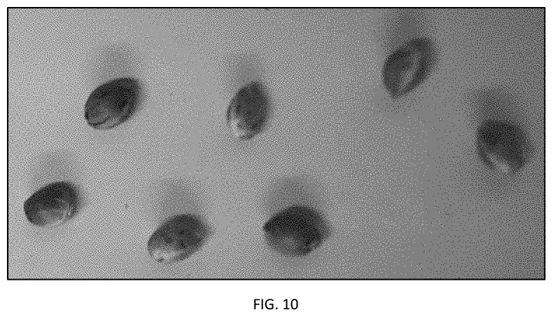

FIG. 10 depicts ‘FRB005’ seeds.

### DETAILED BOTANICAL DESCRIPTION

Some embodiments of the invention relate to a seed from a Cannabis plant designated ‘FRB005’ wherein a representative sample of seed of said plant has been deposited under ______.

Some embodiments of the invention relate to a Cannabis plant, or plant part, tissue, or cell thereof produced by growing the seed of ‘FRB005’, or a descendant thereof. Plant parts can include the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like.

The plants, or plant parts of the invention can display a cannabinoid profile within the ranges set forth in Table 1, as defined herein. The productivity of any given cannabinoid and/or the amounts or ratios of cannabinoids, terpenes, and other plant products can be, by nature, quite variable. The variability can be contributed to by weather, latitude, soil and feeding conditions, pathogens, and numerous other agronomic, horticultural, and biological factors.

Some embodiments of the invention relate to methods of using the plant in a breeding program to produce Cannabis progeny including a cannabinoid profile generally within the ranges as set forth in Table 1. Details of existing Cannabis plant varieties and breeding are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594 778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, R C (Cannabis: Evolution and Ethnobotany 2013), King, J (Cannabible Vols 1-3, 2001-2006), and four volumes of Rosenthal's Big Book of Buds series (2001, 2004, 2007, and 2011), each of which is herein incorporated by reference in its entirety for all purposes.

The present invention also relates to variants, mutants and minor modifications of the seeds, plant parts and/or whole plants of the Cannabis plants of the present invention. Variants, mutants and minor modifications of the seeds, plants, plant parts, plant cells of the present invention can be generated by methods well known and available to one skilled in the art, including but not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference. For more information of mutagenesis in plants, such as agents, protocols, see Acquaah et al. (Principles of plant genetics and breeding, Wiley-Blackwell, 2007, ISBN 1405136464, 9781405136464,) which is herein incorporated by reference in its entirety. Other kinds of modifications practiced in the Cannabis industry, including but not limited to feminization of seeds and/or day-length neutrality/autoflowering are also within the scope of the invention and are within the level of skill in the art to execute.

The present invention also relates to a mutagenized population of the Cannabis plants of the present invention, and methods of using such populations. In some embodiments, the mutagenized population can be used in screening for new Cannabis lines which comprises one or more or all of the morphological, physiological, biological, and/or chemical characteristics of Cannabis plants of the present invention.

In some embodiments, the new Cannabis plants obtained from the screening process comprise one or more or all of the morphological, physiological, biological, and/or chemical characteristics of Cannabis plants of the present invention, and one or more additional or different new morphological, physiological, biological, and/or chemical characteristic.

The present invention also provides any compositions or any products made from or isolated from the plants of the present invention. In some embodiments, the compositions/products comprise an extract of the plants. In some embodiments, the extract can contain a higher percentage of terpenes/terpenoids compared to extract isolated from a control Cannabis plant variety (e.g., an existing variety, such as a recreational Cannabis plant variety). In some embodiments, the invention relates to a smokable or edible product comprising the Cannabis plant, or plant part, tissue, cell, extract, or isolate.

The present invention provides methods of using the Cannabis plants or any parts, any compositions, or any chemicals derived from said plants of the present invention.

In some embodiments, the plants of the present invention can be used to produce new plant varieties. In some embodiments, the plants are used to develop new varieties or hybrids with desired phenotypes or genotypes.

In some embodiments, selection methods, e.g., molecular marker assisted selection, can be combined with breeding methods to accelerate the process. Additional breeding methods known to those of ordinary skill in the art include, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety. The Cannabis genome has been sequenced (Bakel et al., The draft genome and transcriptome of Cannabis sativa, Genome Biology, 12(10):R102, 2011). Molecular makers for Cannabis plants are described in Datwyler et al. (Genetic variation in hemp and marijuana (Cannabis sativa L.) according to amplified fragment length polymorphisms, J Forensic Sci. 2006 March; 51(2):371-5.), Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007), Datwyler et al., (Genetic Variation in Hemp and Marijuana (Cannabis sativa L.) According to Amplified Fragment Length Polymorphisms, J Forensic Sci, March 2006, 51(2):371-375), Gilmore et al. (Isolation of microsatellite markers in Cannabis sativa L. (marijuana), Molecular Ecology Notes, 3(1): 105-107, March 2003), Pacifico et al., (Genetics and marker assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety.

In some embodiments, the invention relates to a Cannabis clone regenerated from the Cannabis plant of descended from the plant, or plant part, tissue, cell or seed of ‘FRB005’ wherein the plant is a clonal descendent.

In some embodiments, the invention relates to a method of producing an F1 Cannabis seed, wherein the method includes crossing the plant with a different Cannabis plant and harvesting the resultant F1 Cannabis seed. In some embodiments, the invention relates to the F1 hybrid Cannabis seed produced by this method. In some embodiments, the invention relates to a F1 hybrid Cannabis plant produced by growing the F1 hybrid Cannabis seed. In some embodiments, the invention relates to a Cannabis clone regenerated from the F1 hybrid Cannabis plant. In some embodiments, the invention relates to a smokable or edible product comprising Cannabis tissue from the F1 hybrid Cannabis plant.

The following detailed description sets forth the distinctive characteristics of ‘FRB005’. Applicant is prepared to make a deposit of seeds or plant tissue. The data which defines these characteristics was collected from seedling plant in the field. Dimensions, sizes, colors, and other characteristics are approximations and averages set forth as accurately as possible. The presented data were collected in Frederick and Longmont, Colo. ‘FRB005’ has not been observed in all possible environments or indoor/outdoor management systems, so variance from the data and the RHS Large Colour Chart (6^{th} edition, 2015) ratings may occur with a change in environment and/or management practices.

### Plant

General:
a. Date and location of 1^{st} self-pollination of clonal ‘Indoor Angie’: Sep. 3, 2019 at Boulder, Colo.
b. Parentage: Female parent: ‘Indoor Angie’ (female parent); Male parent: ‘Indoor Angie’.
c. Data were collected principally at Frederick, Colo. and in the Rocky Mountain Foothills west of Longmont, Colo. in 2020, and to a smaller extent in VT, PA, VA, NC, AL, OH, WI, CA and OR.
d. Botanical classification: Family: Cannabaceae; Species: Cannabis sativa.
e. Growth habit—Very upright, with a mainstem cola that is usually taller than the branch and basal branch colas.
f. Plant life form—Herbaceous tap-rooted annual.
g. Plant propagation—Seed propagated from clonal ‘Indoor Angie’.
h. Propagation ease—Easy.
i. Disease Resistance/Susceptibility: Appears to be intolerant of super wet soils with various fungal root rot pathogens. It did not appear to be affected by high numbers of aphid species present in 2020 Frederick and Longmont, Colo. At present, ‘FRB005’ appears to be no more susceptible to Lepidoptera species caterpillars than any other varieties.
j. Average height—Depends on planting date, density, and fertility/watering practices; with mid-late June transplanting at 121.92-182.88 cm apart within the row, 106.68-137.16 cm tall (3 weeks into flower), with a few rare phenotypes as short as 61 cm and as tall as 178.5 cm.
k. Average spread—Depends on planting date, density, and fertility/watering practices; with mid to late June transplanting at 121.92-182.88 cm apart within the row, 91.44 to 152.4 cm wide, with a few rare phenotypes as low as 30.48 cm and as wide as 169.54 cm.
l. A CBD level of at least 5-8%, e.g., in the range of 5-10% (in dry flower) (in dry flower).
m. A high CBD/THC ratio of at least 20:1, e.g., in the range 20:1 to 25:1 (in dry flower).
n. Dominant terpenes include terpinolene, B-myrcene, and some each of B-caryophyllene, B-pinene, and B-ocimene.
o. Fragrance—Strong pungent aroma with a strong terpene complex, well-liked by growers, with potential for smokable flower.
p. Proportion of hermaphrodite plants—Low, none reported.
q. Proportion of female plants—Very high.
r. Proportion of male plants—Low (3 or less per 5000).

### Stem and Lateral Branch

Mainstem:
a. Average number per plant—1 central, round mainstem, solid interior (not hollow).
b. Average mainstem diameter near soil line—3.2 cm, range of 2.7 to 3.5 cm.
c. Average mainstem diameter where nodes switch from opposite to alternate—2.1 cm, range of 1.5 to 2.2 cm.
d. Average diameter just below mainstem cola—1.2 cm, range of 1.1 to 1.4 cm.
e. Average length of mainstem—114.4 cm, range of 41 to 152 cm.
f. Mainstem texture—Smooth to stipuled.
g. Average length of opposite internodes, lower mainstem—4.7 cm, range of 2.5 to 6.1 cm.
h. Average length of alternate internodes, upper mainstem—3.8 cm, range of 1.3 to 7.0 cm.
i. Depth of lower mainstem grooves (not present on upper or mid-section of mainstem)—Shallow.
j. Mainstem pith in cross section—Thin.
k. Trichome type—Clear, unicellular, non-cystolithic.
l. Mainstem color—Younger sections tend towards RHS 147B (moderate yellow green); older mainstem sections are RHS 144A (strong yellow green), mixed with very shallow groove strips and areas showing colors of RHS 161A (moderate yellow, greyed-yellow group), and RHS 164B and 164C (moderate orange yellow).

Lateral Branch:
a. Average primary branch number—16 opposite, 9 alternate.
b. Average length—Varies from seedling to seedling and along the length of the plant, range of 15.1 to 136.2 cm.
c. Texture—Smooth to stipuled.
d. Color—RHS 147B (moderate yellow green).

### Leaf

General:
a. Leaf color (Top side)—Mostly between NN137A and 137A (week 3-5 flower).
b. Leaf color (Under side)—146B; as leaves fade, a bit closer to 147B.
c. Leaf arrangement—Opposite in early vegetative stages; alternate during flowering.
d. Leaflet shape—Lanceolate, widest at the middle and tapers to a point at the base and apex.
e. Leaflet margins—Serrated, angled towards the apex.
f. Average mainstem leaf width during flowering—14.6 cm, range of 13.9 to 16.5 cm.
g. Average mainstem leaf length during flowering—21.2 cm, range of 17.0 to 24.8 cm.
h. Leaf fragrance—Very faint, but earthy with a hint of pine.
i. Number of leaflets—3-7 leaf tips per leaf, fewer at the top of the plant and around the cola.
j. Central leaflet length—Always longer than side leaflets, 15.2 cm, range of 14.0 to 16.4 cm.
k. Central leaflet width—2.1 cm, range of 1.5 to 2.6 cm.
l. Trichome type (top surface)—Cystolithic hair and capitate sessile.
m. Trichome type (bottom surface)—Unicellular, non-cystolithic.
n. Trichome color (top surface)—Clear.
o. Trichome color (bottom surface)—Clear.
p. Texture on top and bottom surfaces—Smooth to slightly stipuled.
q. Venation type—Midvein in the middle; prominent secondary veins are alternate from midvein; tertiary veins are much less prominent seen much less than the midvein or secondary vein.

### Petiole

General:
a. Mainstem length—6.0 cm, range of 3.0 to 8.4 cm.
b. Mainstem diameter—0.2 cm, range of 0.1 to 0.3 cm.
c. Surface texture—Slightly stipuled.
d. Upper petiole color—RHS 144B.
e. Underside petiole color—RHS 144C.
f. Anthocyanin intensity—On less than 7% of the phenotypes, RHS N77A, slightly to entirely covering both the upper and lower petiole; very rarely covers the underside veins of each leaflet (FIGS. 7 and 8).

### Female Flower

General:
a. Natural flowering season—Early August through September in Colorado, depends on the daylength for each state, and super early (25%) vs. early maturity (75%) phenotypes of ‘FRB005’.
b. Days to flowering—14 days after the short daylength requirement is met.
c. Flower arrangement—Overlapping, stacked, and touching, making a very desirable inflorescence type for smokable flower.
d. Average size of flower—15.3 to 63.7 cm, often 20.3 to 30.5 cm for most flowers.
e. Overall appearance of flower color on the plant in the field—Most are white (whiter than RHS 155D) at week 3-4 flower; a very rare purple phenotype (around RHS 74C) has only been spotted at 1 location in Colorado (2 out of 5000 plants); oddly enough, rare purple petiole phenotypes have only had a white flower appearance so far, in Angie seed grown in the field.
f. Top of mono-leaflet—Similar in color to the bract, depending upon flower week, location, and nutrition, approximating RHS 139A, and/or RHS 147A-B.
g. Mono-leaflet trichomes—Appear to be loaded with all types of upright trichomes on lower and upper leaf surfaces, clear in color; glandular are long stalked capitate, and clear in color.
h. Bract color—Varies depending upon flower week, nutrition, and location, but generally the top is RHS 139A, to RHS 147A-B, and the bottom is RHS 141B-D, and RHS 143B-C.
i. Bract trichomes—Loaded with long stalked capitate, and clear in color.
j. Stigma color—Changes from RHS 145B-C to around RHS 59A-C, 60A-B, and 61A-B after week 3 flowering.
k. Stigma trichomes—Dense in number, and upright, toothed, unicellular, non-cystolithic, and clear in color.

### Seeds

General:
a. Shape—Elliptical to oval longitudinally, flattened at the hilum end and pointed at the apex (FIG. 10); rounded but slightly flattened in width.
b. Length—0.25 cm.
c. Width—0.20 cm.
d. Weight per 1000 seeds—13.24 g.
e. Color of testa—Varies around RHS 199A and 199B (moderate and light olive brown), and RHS 197A (light olive grey); most testa are a solid brownish color, without black (RHS 202A) marbling/speckles or stripes.
f. Marbling—Weak/very low number of seeds with black (RHS 202A) speckles; very rare number of seeds are a solid color with a small amount of black striping.

### Comparison Between Parental and Commercial Varieties

The new C. sativa variety ‘FRB005’ differs from the parental variety ‘Indoor Angie’, by having:

A very small percentage (1 in 1700 plants) of male plants in the field, while ‘Indoor Angie’ has none (Table 1).

A very upright habit and strong root system while ‘Indoor Angie’ almost always leans 30-60 days after transplant. Even after microbursts and sustained winds of over 40-50 mph at Frederick, Colo., ‘FRB005’ was still standing very upright in 2020, while ‘Indoor Angie’ lodged at CO and AL in 2019 with high winds.

A much less even plant height throughout the growing season. Both plant height (Table 1) and width varied widely within locations in 2020 for ‘FRB 005’, while ‘Indoor Angie’ had a much more even height and width distribution (see FIG. 4).

‘FRB 005’ had much greater variability for branch height (FIG. 6, A-D) and more branches/plant (usually at least 12+ per plant). This heavily distinguishes ‘FRB005’ from the original parent, even though many other traits, including leaf color (Table 1) are very similar.

‘FRB 005’ had a few variant phenotypes with varying degrees of purple petiole color (FIGS. 7 and 8). ‘Indoor Angie’ has never been reported to have purple petiole color up to this point in time.

Greater variability in cola length. ‘Indoor Angie’ appeared to be extremely uniform across Alabama and North Carolina in 2019 (Table 1).

‘FRB 005’ had a few, very rare, purple flowering phenotypes (FIG. 9) at week 3 flowering, while the clonal ‘Indoor Angie’ hasn't had any purple flowering phenotypes during week 3 flowering in 2019 or 2020.

Two main phenotypes in ‘FRB 005’ have two different flowering times, 25% that would be best described as “super early flowering, and 75% that can be described as ‘early flowering’ maturity. While the latter group of ‘FRB 005’ had more variability, overall it appeared to be concurrent with flowering for ‘Indoor Angie’, which has had consistent flowering times for all clones in 2019 and 2020.

TABLE 1
Comparison of agronomic and botanical traits of clonal ‘Angie’ vs. ‘FRB005’.

| | Clonal ‘Angie’ | ‘FRB005’ |
|---|---|---|
| Female trait | No males or hermaphrodites | 99.9% female |
| | in the field | |
| Uprightness | Strong tendency to lean, or | Very upright in over 7000 |
| | lodge under the right field | plants at 2 locations in CO, |
| | conditions | and other states around the |
| | | US |
| Plant habit | Christmas tree like to some | Cola is similar in length to |
| | degree; mainstem | the clonal Angie, but the |
| | consistently more prominent | branch length and |
| | and taller than the branches | consequently plant shape, |
| | | vary considerably |
| Flowering Plant Height | 112 to 127 cm with late | Varies considerably between |
| | June/early July transplanting | 61 and 178.5 cm with early |
| | in 2019 | July transplanting in 2020 |
| Mainstem cola | 20 to 30 cm | 15.3 to 45.7 cm |
| Daylength Requirements | Very uniform, typical of | 25% at 14 hrs and 35 |
| for Flowering | clonal varieties, | minutes (Jul. 20, 2020); |
| | 13 hrs and 51 minutes (Aug. | 75% at 13 hrs and 54 |
| | 12, 2019) | minutes (Aug. 10, 2020) |
| Week 3 Flowering | Week of Aug. 26, 2019 in | Variability within each |
| | Frederick and Longmont, CO | class, but approximately |
| | | 25% during week of Aug. 3, |
| | | 2020, and 75% during week |
| | | of Aug. 24, 2020 |
| Upper Leaf Color (RHS | NN137A or NN137B, | Mostly between 137A and |
| Colour Chart)^{†} | depending on location | 137B, a few leaves were |
| | | NN137A |
| Underside Leaf Color | 146B or 146C, depending on | 146B, a few leaves between |
| | the location | 146B and 146C, and at 139C |
| Upper Petiole Color | 144B or 144C, depending on | 144B; few (with NN137A |
| | the location | upper leaf) at N77A; degree |
| | | of N77A purple coverage |
| | | varies from plant to plant |
| Underside Petiole Color | 144C or 145B, depending on | 144C; a few rare ones have |
| | the location | varying degrees of N77A |
| | | purple, only occasionally |
| | | have purple going into the |
| | | veins of the leaflets (see |
| | | FIG. 8) |

^{†}It's unknown at this point, as to whether or not the wildfire smoke and ash that “shortened” the days, affected the top leaf chlorophyll and color ratings in 2020 ‘FRB005’.

What is claimed is:

1. A seed from Cannabis plant designated ‘FRB005’ wherein a representative sample of seed of said plant has been deposited under ______.

2. A Cannabis plant, or plant part, tissue, or cell thereof produced by growing the seed of claim 2, or a descendant thereof.

3. The Cannabis plant, or plant part, tissue, or cell thereof of claim 3 comprising a cannabinoid profile set forth in Table 1.

4. Use of the plant of claim 3 in a breeding program to produce Cannabis progeny comprising a cannabinoid profile set forth in Table 1 and genetic capacity to produce the cannabinoid profile set forth in Table 1 in progeny thereof.

5. The Cannabis plant part of claim 3, wherein said plant part is selected from the group consisting of: stems, trichomes, leaves, and flower buds.

6. The Cannabis plant descended from the plant, or plant part, tissue, cell or seed of claim 3, wherein the plant is a clonal descendent.
